(19) United States
(12) Patent Application Publication
(10) Pub. No.: US 2007/0145352 A1
(43) Pub. Date: Jun. 28, 2007

(54) Endohedral fullerene derivative, proton conductor and fuel cell

(75) Inventors: Yasuhiko Kasama, Miyagi (JP); Kenji Omote, Miyagi (JP); Noboru Kudo, Miyagi (JP)

Correspondence Address: YOUNG & THOMPSON, 745 SOUTH 23RD STREET, 2ND FLOOR, ARLINGTON, VA, 22202, US

(73) Assignee: IDEAL STAR INC., Miyagi (JP)

(21) Appl. No.: 10/577,465

(22) PCT Filed: Oct. 26, 2004
(86) PCT No.: PCT/JP04/15838

§ 371 Date: Feb. 20, 2007

(30) Foreign Application Priority Data
Oct. 28, 2003 (JP) 2003-367820
Jan. 21, 2004 (JP) 2004-013407

(51) Int. Cl. C07D 303/12; H01L 51/00; H01M 4/00

(52) U.S. Cl. 257/40; 549/543; 429/29; 257/E51.02; 977/740

(57) ABSTRACT

With regard to a solid polymer based fuel cell, an electrolyte membrane for conducting proton between a fuel electrode and an air electrode is conventionally made from a material obtained by chemically modifying a hollow fullerene such as C₆₀ by means of a proton dissociable group. However, this fuel cell poses a following problem: since the proton conductivity of the membrane is so low that the internal resistance of battery is increased, which, when big current is extracted, causes the electromotive force to be reduced. The electrolyte membrane is made of a material obtained by chemically modifying an endohedral fullerene doped with an atom whose electronegativity is equal to or higher than 3, by means of a proton dissociable group, or a material made from an endohedral fullerene doped with an atom whose electronegativity is equal to or lower than 1. The membrane ensures the improved proton conductivity and reduced internal resistance of battery, as compared with the conventional electrolyte membrane made from a material obtained by chemically modifying a hollow fullerene by means of a proton dissociable group.

### TECHNICAL FIELD

The present invention relates to a solid polymer-based fuel cell utilizing, as a fuel, hydrogen or a hydrocarbon compound such as methanol, and to a proton conductor which will be used as a material of an electrolyte membrane serving as an element of such a solid polymer-based fuel cell.

The invention further relates to a gas detector for detecting hydrogen or a hydrocarbon compound such as methane, and to a proton conductor which will be used as a material of an electrolyte membrane serving as an element of such a gas detector.

### BACKGROUND ART

Patent Document 1: Japanese Unexamined Patent Application Publication No. 2002-216803

Patent Document 2: Japanese Unexamined Patent Application Publication No. 2002-193861

Non-patent Document 1: “Latest innovation of nanotechnology,” Nikkei Science, supplementary vol. 138, p. 31

Non-patent Document 2: Paul R. Birkett et al., Nature (1992), 357, 479

Instead of the conventional petroleum-based energy source whose resource will be exhausted sooner or later, and which is responsible for the pollution of air, alternative clean energy sources such as solar and wind energies have attracted attention, and the practical utilization of such alternative energy sources has been advanced as represented by electrical generation based on solar and wind energies. However, electricity derived from such alternative energy sources can not be preserved as it is. To cope with this inconvenience, a following promising scheme has been proposed as a next-generation electricity preservation/supply system: electricity derived from solar and wind energies is used to decompose water to produce hydrogen, and the hydrogen is used as a fuel of a fuel cell to generate electricity as needed. Indeed, research and development efforts for the fabrication of such a fuel cell which will be applied in the field such as the manufacture of electric cars, home-use electric generators, small fuel cell-based batteries for mobile phones, etc. have been advanced.

### DISCLOSURE OF THE INVENTION

Problems to be Solved by the Invention

An electrolyte membrane serving as an element of a fuel cell consists of a substance capable of transmitting hydrogen ions (protons) from a negative electrode (anode) to a positive electrode (cathode). The selection of a substance suitable for the electrolyte membrane is very important for the improved performance of a fuel cell, as well as the selection of substances suitable for the electrodes. Currently, as the electrolyte membrane, fluorine resin-based membranes have been put into practice. However, the development of an electrolyte membrane made of a hollow fullerene is now under way because, if it were possible to produce such an electrolyte membrane, the membrane would obviate the need for moisturization, and enable the production of a thinner membrane capable of working over a wider temperature range, as compared with the currently available fluorine resin-based electrolyte membrane. However, the fullerene-based electrolyte membrane has a low conductivity being equal to one hundredth to one thousandth of that of the conventional fluorine-based electrolyte membrane, which leads to the increased internal resistance. Thus, even if a fuel cell incorporating such an electrolyte membrane is put into use, extraction of a large current from the cell would lead to the dropping of its electromotive force. (Non-patent Document 1).

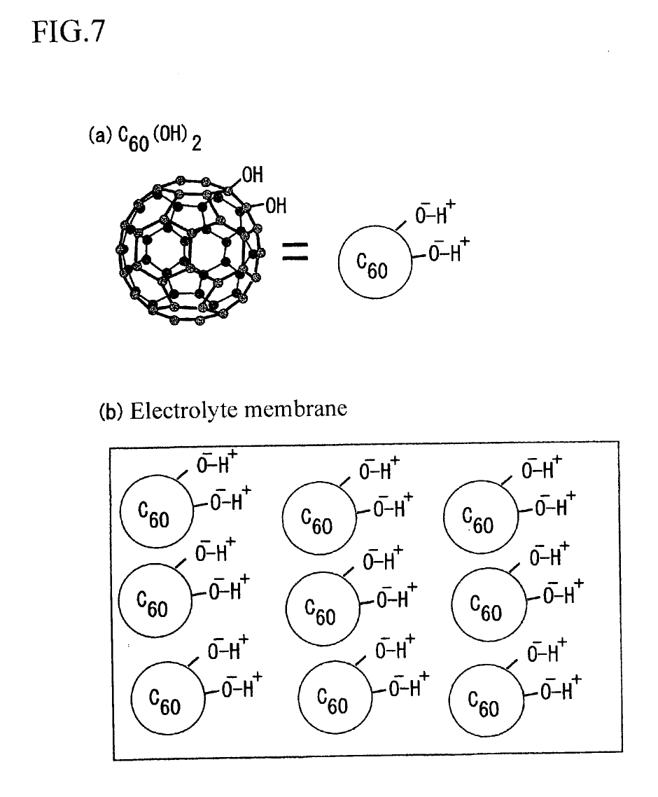

FIGS. 7(a) and 7(b) illustrate a proton conductor comprised of a fullerene derivative obtained by chemically modifying a conventional hollow fullerene by means of a proton dissociable group. For example, FIG. 7(a) illustrates a fullerene derivative obtained by chemically modifying a fullerene or a C₆₀ compound where 60 carbon atoms are connected to each other in the form of a closed cage, so that the modified fullerene has proton dissociable groups, for example, OH groups. For the convenience of description, a fullerene molecule is represented by a circle. FIG. 7(b) represents a proton conductor (electrolyte membrane) comprised of a fullerene derivative as shown in FIG. 7(a). (Patent Document 1).

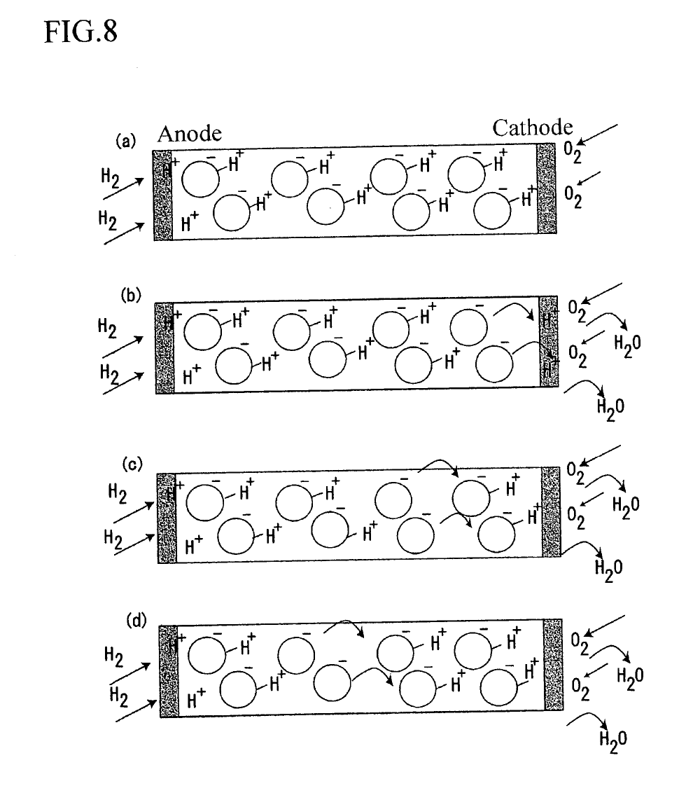

FIGS. 8(a) to 8(d) illustrate how protons are conducted through a proton conductor comprised of a conventional fullerene derivative like the one shown in FIG. 7. Referring to FIG. 8, a fuel cell includes an electrolyte membrane between an anode and a cathode and supplies hydrogen to the anode and oxygen or air containing oxygen to the cathode. On the anode, hydrogen is converted, under the action of a catalyst coated on the anode, into protons, while on the cathode a reaction occurs where oxygen combines with protons to turn into water. Accordingly, the concentration of protons on the anode is increased while the concentration of protons on the cathode is decreased. If the electrolyte membrane has a property to transmit protons, protons will move by diffusion from the anode to the cathode. If the electrolyte membrane is made of a fullerene derivative obtained by adding OH groups to a conventional hollow fullerene like the one shown in FIG. 7(b), the following problem will arise. Let's assume that the hydrogen atom of a proton dissociable group is dissociated from the group to become a free positive ion. Then, there will arise an attractive force between the positive ion and the oxygen atom of the same group because the oxygen atom is then negatively charged, which will inhibit the dissociation of the hydrogen atom. On the whole, this will result in the membrane having an insufficient conductivity towards protons.

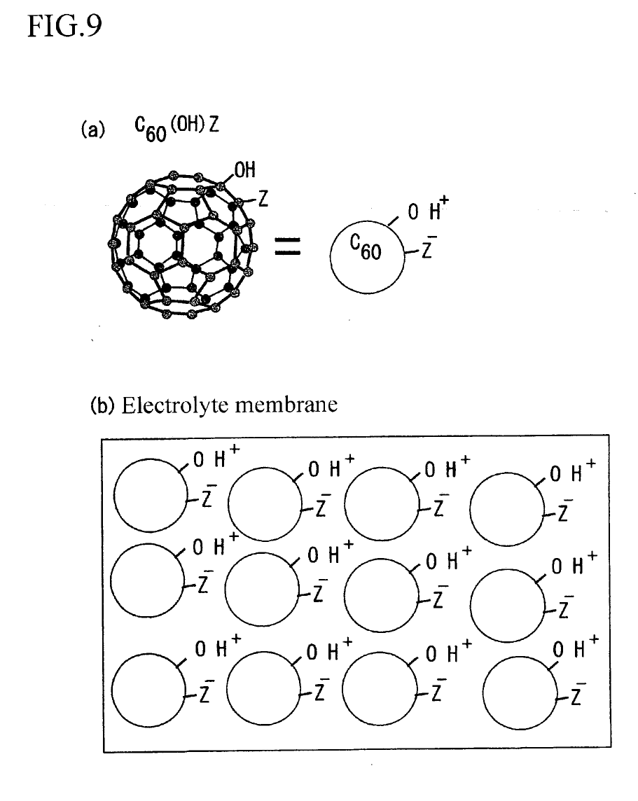

FIGS. 9(a) and 9(b) illustrate a proton conductor comprised of a fullerene derivative obtained by chemically modifying a conventional hollow fullerene such that the modified fullerene has a proton dissociable group and an electron attracting group. In FIG. 9, the electron attracting group is represented by —Z. Suitable electron attracting groups may include, for example, —NO₂, —CN, —F, —COOR, etc. (Patent Document 1).

If an electrolyte membrane consists of a proton conductor made of a fullerene derivative having an electron attracting group attached thereto in addition to an OH group like the one shown in FIG. 9, the following problem will arise. Let's assume that the hydrogen atom of a proton dissociable group is dissociated from the group to become a proton. Then, since the electron of the hydrogen atom is attracted by the electron attracting group, dissociation of the proton from the group will be facilitated. However, the dissociated proton is also attracted by the electron attracting group which is now negatively charged, which will interfere with the mobility of the proton. On the whole, this will result in the membrane having a rather low conductivity towards protons like the one described above.

Means for Solving the Problems

The present inventors tried to find a proton conductor including an endohedral fullerene derivative or an endohedral fullerene having a high conductivity towards protons, and to employ such a proton conductor as a material of the electrolyte membrane of a fuel cell.

Aspect (1) of the present invention relates to an endohedral fullerene derivative obtained by chemically modifying an endohedral fullerene doped with an atom whose electronegativity is 3 or higher, by means of a proton dissociable group.

Aspect (2) of the invention relates to an endohedral fullerene derivative as described with respect to aspect (1) wherein the proton dissociable group is any one selected from the group comprising —OH, —OSO₃H, —COOH, —SO₃H, and —OPO(OH)₂.

Aspect (3) of the invention relates to a proton conductor comprised of an endohedral fullerene derivative as described with respect to aspect (1) or (2).

Aspect (4) of the invention relates to a proton conductor comprised of an endohedral fullerene doped with an atom whose electric negativity is equal to or less than 1.

Aspect (5) of the invention relates to a proton conductor comprised of a polymerized endohedral fullerene derivative obtained by polymerizing an endohedral fullerene derivative as described with respect to aspect (3), or comprised of a polymerized endohedral fullerene obtained by polymerizing an endohedral fullerene as described with respect to aspect (4).

Aspect (6) of the invention relates to a fuel battery comprising a stack of cells each comprising a fuel electrode, an electrolyte membrane including a proton conductor as described with respect to any one of aspects (3) to (5), and an air electrode.

Aspect (7) of the invention relates to a gas detector having a gas detection unit comprising a stack of cells each comprising an anode catalyst, an electrolyte membrane including a proton conductor as described with respect to any one of aspects (3) to (5), and a cathode catalyst.

Aspect (8) of the invention relates to a method for determining the concentration of gas such as hydrogen or hydrocarbon gas using a gas detector as described with respect to aspect (7).

Aspect (9) of the invention relates to a leak detector having a gas detection unit comprising a stack of cells each comprising an anode catalyst, an electrolyte membrane including a proton conductor as described with respect to any one of aspects (3) to (5), and a cathode catalyst.

Aspect (10) of the invention relates to a leak detection method for checking whether any leak occurs in a device to be tested and for identifying the site of leak if any leak is detected, the method comprising employing hydrogen as a probe gas, and using a leak detector as described with respect to aspect (9).

Effect of the Invention

(1) With regard to a proton conductor comprised of an endohedral fullerene derivative obtained by chemically modifying an endohedral fullerene doped with an atom whose electric negativity is equal to or higher than 3, by means of a proton dissociable group such as —OH, —OSO₃H, —COOH, —SO₃H, or —OPO(OH)₂, when such a proton conductor is used as an electrolyte membrane of a fuel cell, dissociation of proton from a proton dissociable group occurs easily because proton is attracted by the dopant atom, but the attractive force exerted by the dopant atom is small because of the negatively charged dopant atom being enclosed in the cage of fullerene. Because of these properties, protons can freely move in the electrolyte membrane which results in the increased proton conductivity of the electrolyte membrane.

(2) With regard to a proton conductor comprised of an endohedral fullerene doped with an atom whose electronegativity is equal to or lower than 1, the dopant atom will become a positive ion by giving off an electron to the fullerene cage. The fullerene cage becomes negatively charged because of receiving an electron.

However, the electron is not localized upon a specific carbon, and thus the attractive force exerted by the fullerene cage towards proton is comparatively weak. Therefore, protons can move freely being driven by a comparatively low thermal energy through the electrolyte membrane where proton conducting elements are densely packed, which results in the increased proton conductivity of the electrolyte membrane.

(3) With regard to a proton conductor comprised of a polymerized endohedral fullerene or a polymerized endohedral fullerene derivative obtained by polymerizing an endohedral fullerene or an endohedral fullerene derivative, it is excellent in its mechanical strength.

(4) A fuel battery incorporating an electrolyte membrane based on a proton conductor of the invention is more advantageous than a conventional battery incorporating a fluorine resin-based electrolyte membrane, because the inventive membrane obviates the need for moisturization, and enables the production of a thinner membrane capable of working over a wider temperature range, and further reduces the internal resistance sufficiently low as to inhibit the dropping of voltage even if big current is extracted.

(5) According to a gas detector incorporating an electrolyte membrane based on a proton conductor of the invention, it is possible to determine the concentration of hydrogen or hydrocarbon at high sensitivity.

(6) According to a leak detector incorporating an electrolyte membrane based on a proton conductor of the invention, it is possible to check a vacuum unit or gas range for leak at high sensitivity by using, for example, hydrogen as probe gas.

### BRIEF DESCRIPTION OF THE INVENTION

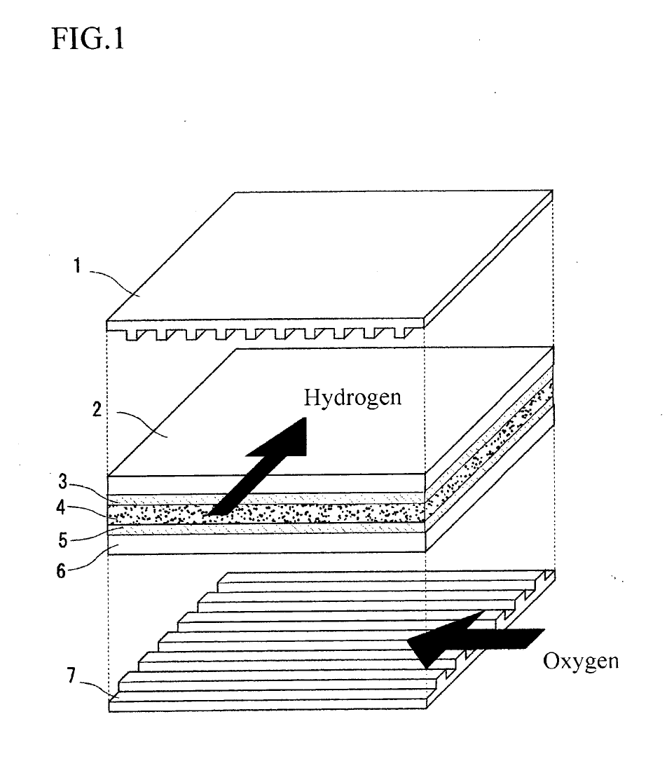

FIG. 1 is a perspective view for showing the structure of a single cell constituting a solid polymer-based fuel battery.

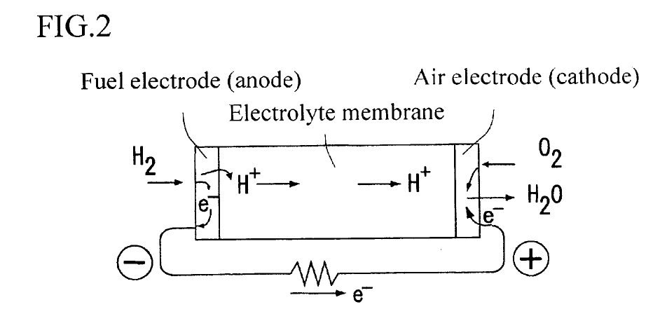

FIG. 2 illustrates how electricity is generated by a hydrogen direct transfer type fuel cell with a solid polymer-based electrolyte membrane.

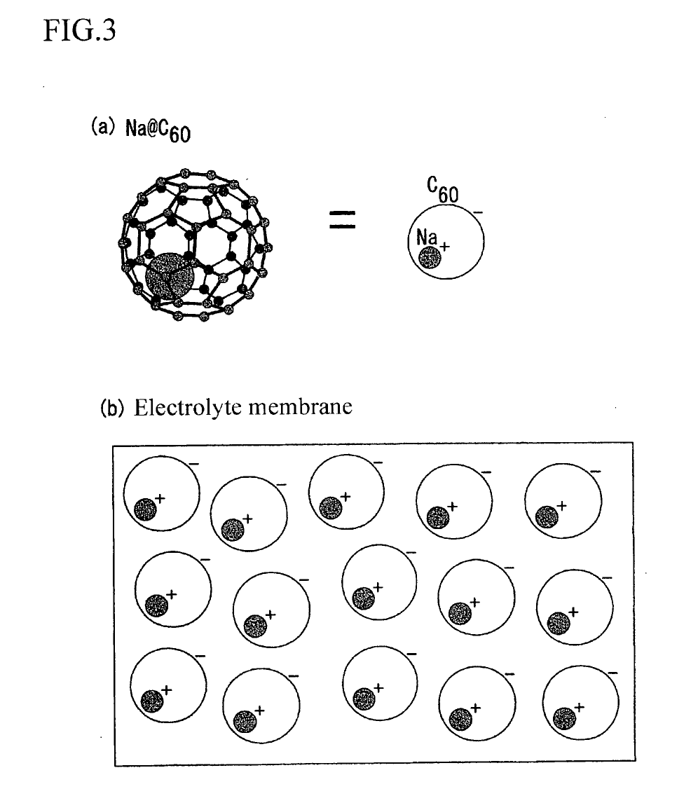

FIGS. 3(a) and 3(b) illustrate a proton conductor comprised of an inventive endohedral fullerene doped with an atom whose electronegativity is equal to or less than 1.

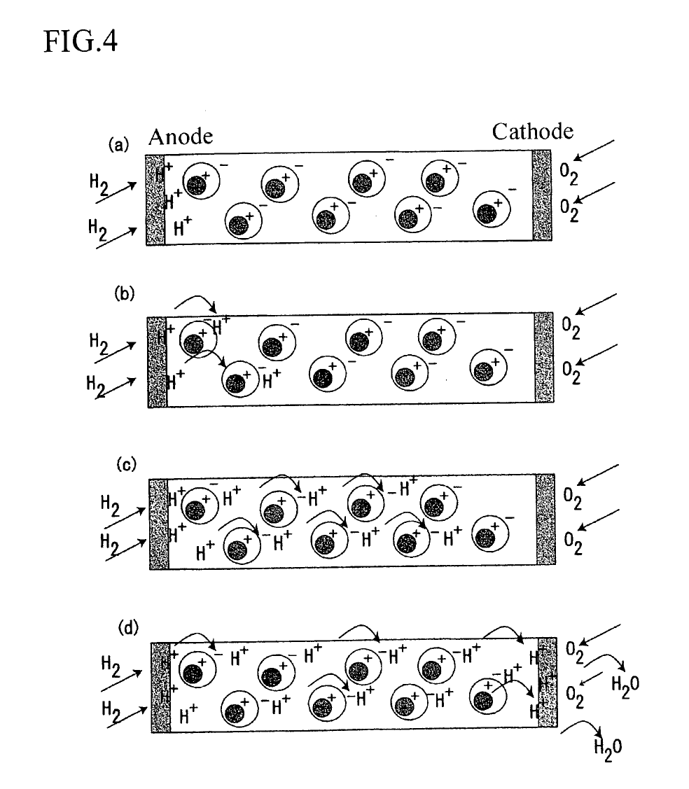

FIGS. 4(a) to 4(d) illustrate how protons are conducted through a proton conductor comprised of an inventive endohedral fullerene enclosing an atom whose electronegativity is equal to or lower than 1.

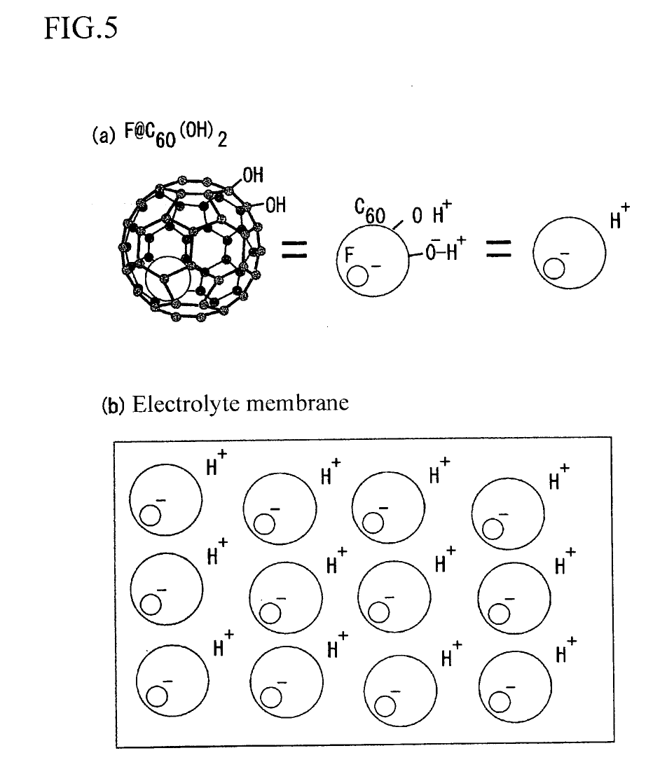

FIGS. 5(a) and 5(b) illustrate the operation of a proton conductor comprised of an endohedral fullerene derivative obtained by chemically modifying an inventive endohedral fullerene enclosing an atom whose electronegativity is equal to or higher than 3, by means of a proton dissociable group.

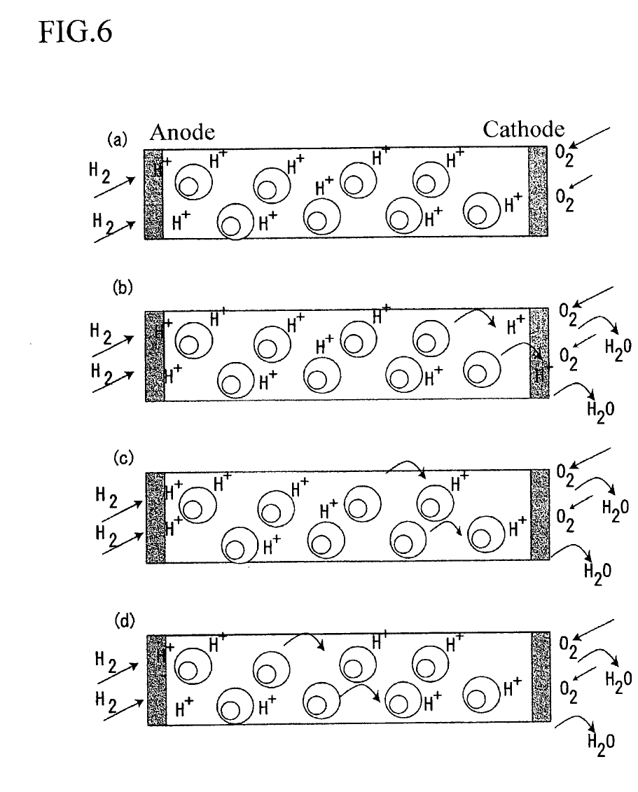

FIGS. 6(a) to 6(d) illustrate how protons are conducted through a proton conductor comprised of an endohedral fullerene derivative obtained by chemically modifying an inventive endohedral fullerene enclosing an atom whose electronegativity is equal to or higher than 3, by means of a proton dissociable group.

FIGS. 7(a) and 7(b) illustrate a proton conductor comprised of a fullerene derivative obtained by chemically modifying a conventional hollow fullerene by means of a proton dissociable group.

FIGS. 8(a) to 8(d) illustrate how protons are conducted through a proton conductor comprised of an endohedral fullerene derivative obtained by chemically modifying a conventional hollow fullerene by means of a proton dissociable group.

FIGS. 9(a) and 9(b) illustrate a proton conductor comprised of a fullerene derivative obtained by chemically modifying a conventional hollow fullerene by means of a proton dissociable group and an electron attracting group.

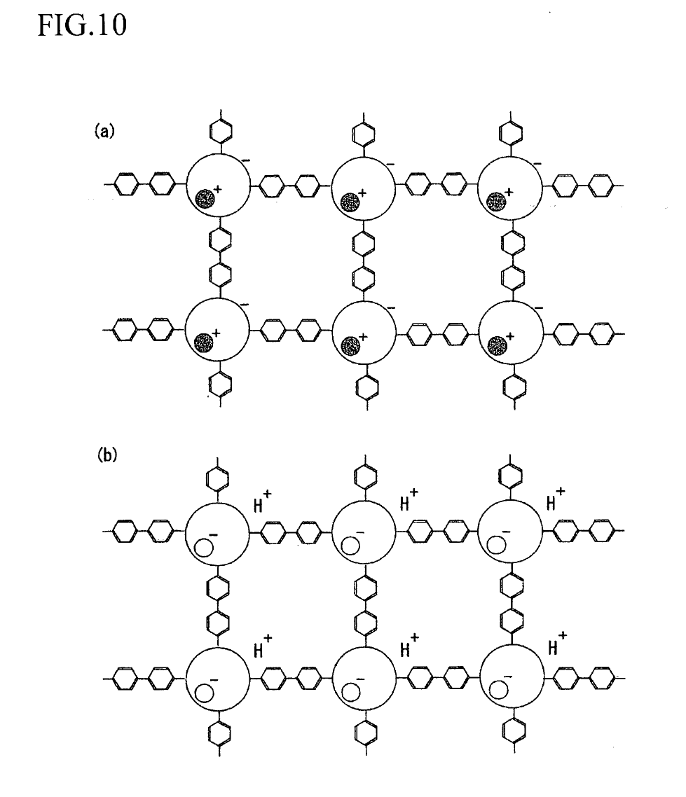

FIG. 10(a) illustrates a proton conductor comprised of a polymerized endohedral fullerene obtained by polymerizing an inventive endohedral fullerene doped with an atom whose electronegativity is equal to or lower than 1 through a linker comprising an aromatic group. FIG. 10(b) illustrates a proton conductor comprised of a polymerized endohedral fullerene derivative obtained by chemically modifying an inventive endohedral fullerene doped with an atom whose electronegativity is equal to or higher than 3 by means of a proton dissociable group, and then polymerizing the resulting endohedral fullerene derivative through a linker comprising an aromatic group.

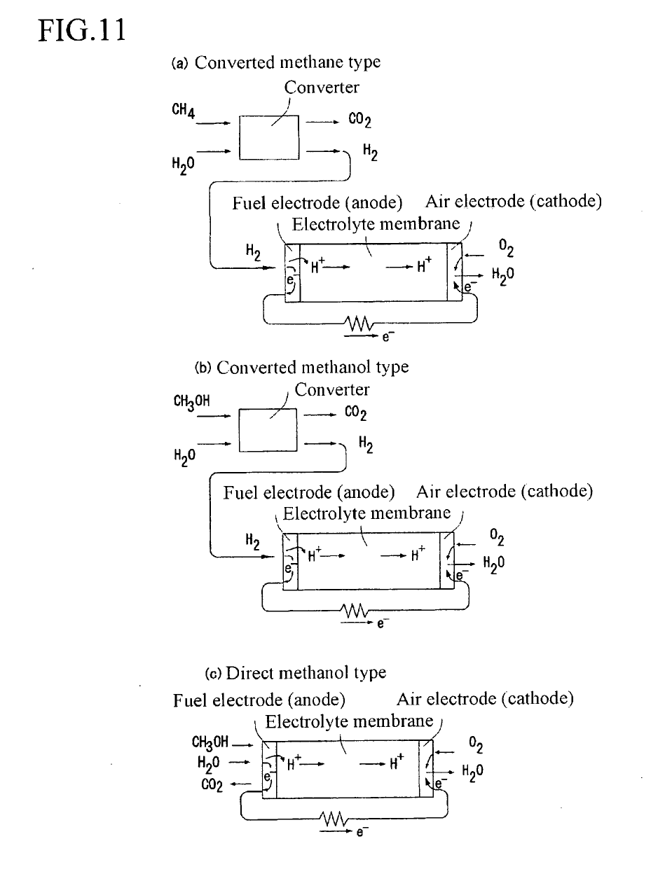

FIGS. 11(a) to 11(c) illustrates the electric generation of three different types of solid polymer-based fuel cells, that is, converted methane type, converted methanol type and direct methanol type, respectively.

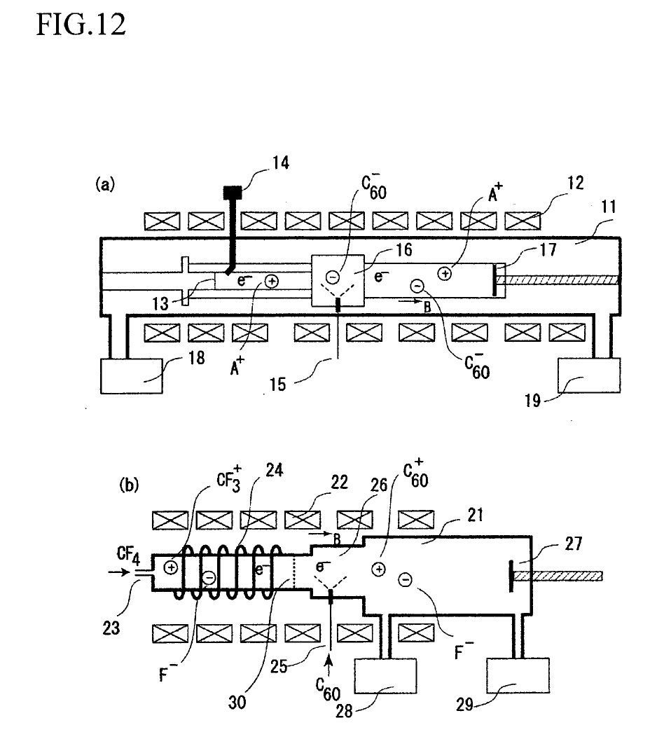

FIG. 12(a) is a sectional view of a device for manufacturing endohedral fullerenes by utilizing plasma generated as a result of contact ionization. FIG. 12(b) is a sectional view of a device for manufacturing endohedral fullerenes by utilizing plasma generated as a result of RF induction.

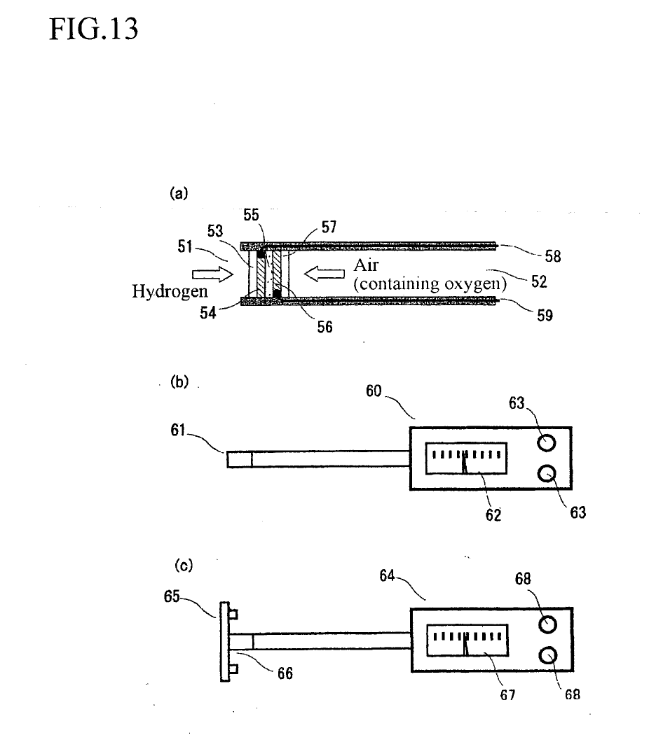

FIG. 13(a) is a sectional view of a gas detection unit of a hydrogen gas detector of the invention. FIGS. 13(b) and 13(c) represent schematic overviews of first and second embodiments of the gas detector of the invention, respectively.

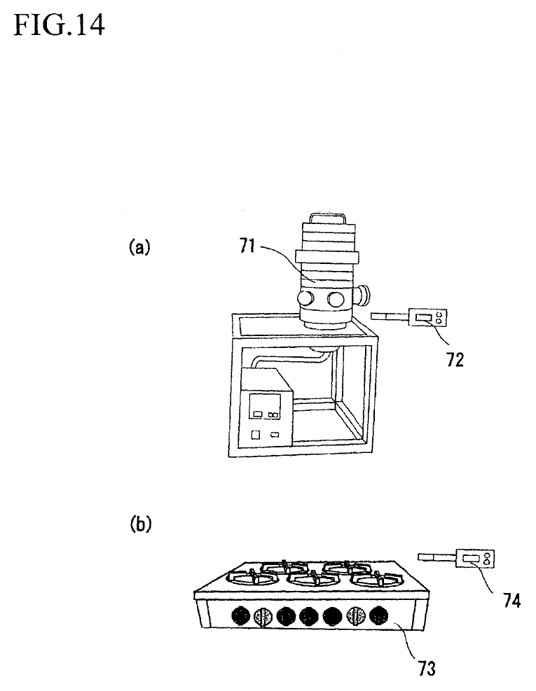

FIG. 14(a) is a perspective view of a gas detector of the invention being used for checking a vacuum unit for leak. FIG. 14(b) is a perspective view of a gas detector of the invention being used for checking a gas range for leak.

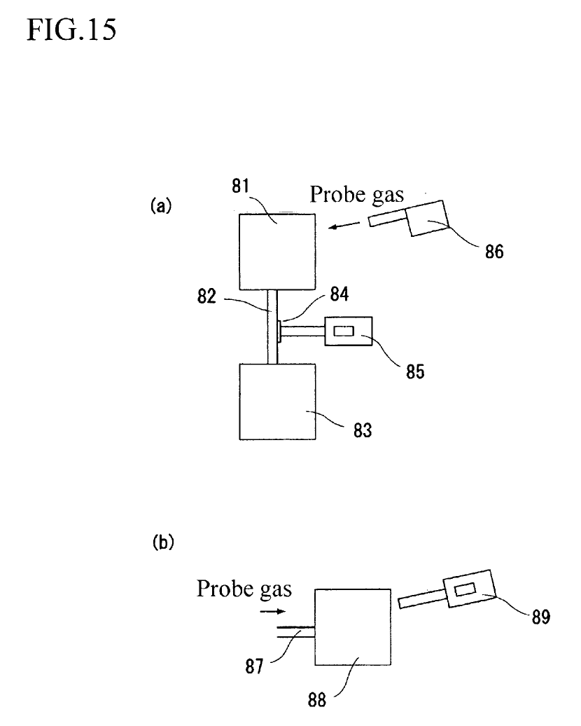

FIG. 15(a) illustrates how a gas detector of the invention can be used for checking a vacuum unit for leak by probe gas flushing. FIG. 15(b) illustrates how a gas detector of the invention can be used for checking a vacuum unit for leak by internal pressurization.

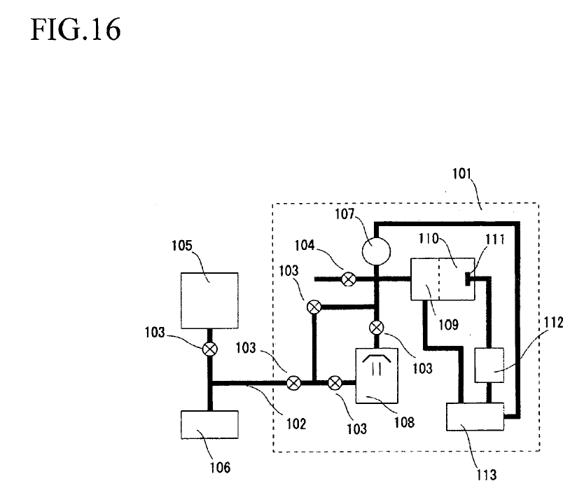

FIG. 16 is a block diagram for illustrating the organization of a conventional helium leak detector.

### REFERENCE NUMERALS

1, 7: Separator
2, 6: Porous support
3: Anode catalyst
4: Polymer-based electrolyte membrane
5: Cathode catalyst
11, 21: Endohedral fullerene manufacturing device
12, 22: Electromagnetic coil
13: Hot plate
14: Oven for vaporizing atom to be doped
15, 25: Oven for sublimating fullerenes
16, 26: Cylinder for resublimation
17, 27: Deposition substrate
18, 19, 28, 29: Vacuum pump
23: Inlet pipe for introducing gas of an atom to be doped
24: RF induction coil
30: Grid electrode
51: Gas detection opening
52: Air supply opening
53, 57: Porous support
54: Anode catalyst
55: Polymer-based electrolyte membrane
56: Cathode catalyst
58: Anode lead
59: Cathode lead
60, 64: Gas detector
61, 66: Gas detection unit
62, 67: Display unit
63, 68: Operational switch
65: Flange
71: Vacuum unit
72, 74: Leak detector
73: Gas range
81, 88: Vacuum unit (subject to be tested)
82: Exhaust pipe
83: Vacuum pump
84: Flange
85, 89: Leak detector
86: Probe gas blower
87: Probe gas supply pipe
101: Leak detector
102: Distributor pipe
103: Valve
104: Leak valve
105: Vacuum unit (subject to be tested)
106: Rotary pump
107: Vacuum meter
108: Diffusion pump
109: Ion source
110: Analysis tube
111: Ion collector
112: Amplifier
113: Power supply

### BEST MODE FOR CARRYING OUT THE INVENTION

The definitions of the terms as used herein will be given below, and the embodiments best representing the present invention will be described.

The term “fullerene” refers to a hollow carbon cluster substance in which carbon atoms represented by formula Cₙ (n=60, 70, 76, 78, . . . ) are connected to each other in the form of a closed cage, and it includes, for example, C₆₀ or C₇₀. According to the present definition, the term “fullerene” includes not only a pure population comprising a single fullerene isomer, but also a population comprising “fullerene mixtures” or “fullerene conjugates.”

The term “fullerene mixture” refers to a carbon cluster population comprising two or more different fullerene isomers. When fullerenes are produced by resistance heating or arc discharging, the percent weights of fullerene isomers are generally as follows: 70 to 85% for C₆₀, 10 to 15% for C₇₀, and the rest for higher-order fullerenes such as C₇₆, C₇₈, C₈₄, etc. When fullerenes are produced by the combustion method, the percent weights of fullerene isomers are practically the same: the summed percent weight of C₆₀ and C₇₀ is higher than the summed percent weight of higher-order fullerene isomers. Thus, C₆₀ and C₇₀ are more readily available and cheaper than other higher-order fullerene isomers. Not only isolated C₆₀ or C₇₀ fullerenes but also fullerene mixtures comprising C₆₀ and C₇₀ isomers may be obtained, for example, from Frontier Carbon or other providers.

The term “fullerene conjugate” refers to a carbon cluster substance comprising two or more fullerenes linked together as represented by a fullerene dimer or fullerene trimer.

The term “atom-doped” fullerene refers to a fullerene enclosing an atom other than carbon within the hollow space of its cage-like structure. The number of doped atom(s) may be one or two or more.

The term “hollow fullerene” refers to a fullerene which does not enclose any atom within its hollow space.

The term “endohedral fullerene” refers to a fullerene enclosing an atom within the hollow space of its cage-like structure. If a fullerene conjugate comprises two or more fullerenes, it is not always necessary for the fullerenes to contain an atom within their follow space (for example, if a fullerene conjugate is a fullerene dimer, one monomer unit may contain an atom within its hollow space while the other may not contain an atom within its hollow space).

The predicate “comprise” includes the implication represented by the predicate “consist solely of” and the implication represented by the predicate “include.” Therefore, for example, a proton conductor according to aspect (3) of the invention may include a component other than an endohedral fullerene derivative.

The term “ionization energy” refers to an energy necessary for exciting an electron at the outermost shell of a neutral atom to a level sufficiently high to purge the electron as a free electron, thereby turning the atom now deprived of one electron into a positive ion. It is difficult to turn an atom into a positive ion, if the atom requires a high energy for ionization.

The term “electron affinity” refers to an energy released by an atom, when a free electron puts itself in an unfilled electron orbital of the atom, thereby turning the atom into a negative ion. It is easy to turn an atom into a negative ion, if the atom has a high electron affinity.

If a given atom has a high ionization energy and high electron affinity (in terms of absolute values), the atom is ready to become a negative ion. The readiness with which an atom becomes a negative ion (ability to attract an electron) is called “electronegativity.” The electronegativity of an atom can be expressed by an average of the absolute value of its ionization energy and the absolute value of its electron affinity.

Atoms whose electronegativity is equal to or higher than 3 include F, O, Cl, N, etc. Atoms whose electronegativity is equal to or lower than 1 include Cs, Rb, K, Ba, Na, Sr, Ca, Li, etc.

The term “proton dissociable group” refers to a functional group where a proton can be dissociated as a result of ionization. The proton dissociable group may include, for example, —OH, —OSO₃H, —COOH, —SO₃H, and —OPO(OH)₂.

The term “endohedral fullerene derivative” refers to a modified endohedral fullerene obtained by chemically modifying an endohedral fullerene by means of a functional group such as a proton dissociable group.

The term “fuel electrode” refers to an electrode which, in a fuel cell, serves as an electrode for supplying a fuel such as hydrogen or hydrocarbon. This electrode is also called a negative electrode (anode) because electrons are emitted from this electrode.

The term “air electrode” refers to an electrode which, in a fuel cell, serves as an electrode for supplying oxygen or air containing oxygen. This electrode is also called a positive electrode (cathode) because this electrode receives electrons.

(Principle Underlying the Electric Generation of a Hydrogen Direct Transfer Type Fuel Cell)

FIG. 1 is a perspective view for showing the structure of a single cell constituting a solid polymer-based fuel battery. A single cell of a fuel battery comprises a proton-conductive polymer-based electrolyte membrane 4 inserted between an anode and a cathode where the anode consists of an porous support 2 and anode catalyst 3 while the cathode consists of another porous support 6 and cathode catalyst 5, the porous supports 2, 6 being further enclosed by separators 1, 7, respectively. The theoretical electromotive force of a fuel cell is 1.23V. If a higher electromotive force is required, it is only necessary to prepare a fuel battery comprising a stack of a desired number of electric cells. The electrode may be obtained by coating the surface of a porous support with a carbon carrier in which an electrode catalyst such as a precious metal, e.g., platinum in the form of a powder is uniformly dispersed.

FIG. 2 illustrates how electricity is generated by a hydrogen direct transfer type fuel cell with a solid polymer-based electrolyte membrane. Hydrogen is allowed to flow through a groove formed on the separator in the vicinity of anode while oxygen or air containing oxygen is allowed to flow through a groove on the separator in the vicinity of cathode. Then, the following reactions occur.

On the anode side: 2H₂-->4H⁺+4e⁻

On the cathode side: 4H⁺+O₂+4e⁻-->2H₂O

Protons generated at the anode migrate through the proton-conductive electrolyte membrane towards the cathode. At the same time, electrons generated at the anode flow through an external circuit towards the cathode.

(Proton Conductor)

The proton conductor according to the present invention may take two or more different structures depending on the type of its constitutive fullerene derivatives or fullerenes. Proton conductors having different constitutions according to the invention will be described in detail with reference to the attached drawings.

First Illustrative Embodiment

FIGS. 3(a) and 3(b) illustrate a proton conductor comprised of an endohedral fullerene doped with an atom whose electronegativity is equal to or less than 1. FIG. 3(a) shows the structure of a C₆₀ molecule doped with Na or an alkali metal as an illustrative example of an endohedral fullerene doped with an atom whose electronegativity is equal to or less than 1. As seen from the figure, an Na atom represented by a closed circle is enclosed in the cage of C₆₀ molecule. For the convenience of description, a fullerene doped with Na is represented by an open circle representing the fullerene with a closed circle representing Na in its closed space. It has been known that a fullerene, when it is brought into the vicinity of an atom whose electronegativity is equal to or lower than 1, readily takes an electron from the atom, whereas when it is brought into the vicinity of an atom whose electronegativity is equal to or higher than 3, it readily confers an electron to the atom. Since the dopant atom of the fullerene under study is Na whose electronetativity is sufficiently small, the dopant atom readily confers an electron to the fullerene to cause the fullerene to be negatively charged, whereas the dopant atom itself, because of its giving off an electron, comes to be positively charged. The negative charge thus generated is not localized to a specific carbon atom(s) constituting the C₆₀ molecule, but spreads on a comparatively wide area over the cage-like C₆₀ molecule.

FIG. 3(b) shows an electrolyte membrane comprised of a material where Na doped fullerenes are densely packed. FIGS. 4(a) to 4(d) illustrate how protons are conducted through a proton conductor comprised of an Na doped fullerene according to the invention. Referring to FIG. 4, the fuel cell comprises an electrolyte membrane inserted between an anode and a cathode, and supplies hydrogen to the anode and oxygen or air containing oxygen to the cathode. Following reactions occur on the respective electrodes: on the anode, hydrogen turns, under the action of a catalyst coated on the anode, into protons, while on the cathode oxygen combines with proton to turn into water. Accordingly, the concentration of protons on the anode is increased while the concentration of protons on the cathode is decreased. Then, protons will move by diffusion from the anode to the cathode. During the passage through electrolytes, protons are associated with the negatively charged surface of endohedral fullerenes. However, the attractive force between a proton and negative charge spread over a cage-shaped fullerene is so weak that the proton can easily escape from the restriction imposed by the fullerene to migrate to an adjacent fullerene. Thus, an electrolyte membrane comprised of a fullerene derivative according to the invention has a high conductivity towards protons.

In the above embodiment where a proton conductor is comprised of an endohedral fullerene doped with an atom whose electronegativity is equal to or lower than 1, description was given taking Na-doped C₆₀ as an example of such an endohedral fullerene. However, if the Na-doped C₆₀ is substituted for an endohedral fullerene with another dopant atom whose electronegativity is equal to or lower than 1, for example, Cs, Rb, K, Ba, Sr, Ca, or Li, the same advantage of increased proton conductivity will be obtained as in the above embodiment based on Na-doped C₆₀. Or, if the fullerene molecule C₆₀ itself is substituted for another fullerene molecule, for example, any one of the fullerenes represented by Cₙ (n=70, 76, 78, . . . ), the same advantage of increased proton conductivity will be obtained as in the above embodiment based on C₆₀.

Second Illustrative Embodiment

FIGS. 5(a) and 5(b) illustrate the operation of a proton conductor comprised of an endohedral fullerene derivative obtained by chemically modifying an inventive endohedral fullerene enclosing an atom whose electronegativity is equal to or higher than 3, by means of a proton dissociable group. FIG. 5(a) shows the structure of a molecule obtained by chemically modifying a C₆₀ molecule enclosing, for example, F as an atom whose electronegativity is equal to or higher than 3, so that the modified molecule has two —OH groups. The C₆₀ molecule encloses, in its cage, F represented by a small open circle.

For the convenience of description, an F-doped C₆₀ molecule will be represented by a small open circle for F enclosed by a large open circle for C₆₀. The dopant atom F, because of its high electronegativity, robs an electron from the hydrogen atom of an —OH group to be negatively charged, while the hydrogen atom, being deprived of electron, turns into a proton.

FIG. 5(b) shows an electrolyte membrane comprised of a material where F doped fullerenes are densely packed. FIGS. 6(a) to 6(d) illustrate how protons are conducted through a proton conductor comprised of an F doped fullerene of the invention which has a proton dissociable group as a result of chemical modification. Referring to FIG. 6, the fuel cell comprises an electrolyte membrane inserted between an anode and a cathode, and supplies hydrogen to the anode and oxygen or air containing oxygen to the cathode. Following reactions occur on the respective electrodes: on the anode, hydrogen turns, under the action of a catalyst coated on the anode, into protons, while on the cathode oxygen combines with proton to turn into water. Accordingly, the concentration of protons on the anode is increased while the concentration of protons on the cathode is decreased. Then, protons will move by diffusion from the anode to the cathode.

Each of the fullerene derivatives constituting the electrolyte membrane has its own dissociable group. Protons are consumed from fullerenes adjacent to the cathode, to those fullerenes deprived of protons, protons are supplied by adjacent fullerenes on the opposite side, and this process is repeated from the cathode side towards the anode. Thus, protons migrate from the anode to the cathode. For each fullerene derivative, negative charge interfering with the migration of proton by attracting the proton towards itself is enclosed within the cage of the fullerene derivative, and thus it can not exert strong attractive force onto the proton. Thus, an electrolyte membrane comprised of a fullerene derivative of the invention obtained by chemically modifying an endohedral fullerene enclosing an atom whose electronegativity is equal to or higher than 3, by means of a proton dissociable group can have a high conductivity towards protons.

In the above embodiment where a proton conductor is comprised of a fullerene derivative obtained by chemically modifying an endohedral fullerene enclosing an atom whose electronegativity is equal to or higher than 3, by means of a proton dissociable group, description was given taking F-doped C₆₀(OH)₂ as an example of such a fullerene derivative. However, if the F-doped C₆₀ is substituted for an endohedral fullerene with another dopant atom whose electronegativity is equal to or higher than 3, for example, O, Cl or N, the same advantage of increased proton conductivity will be obtained as in the above embodiment based on F-doped C₆₀. Or, if the fullerene molecule C₆₀ itself is substituted for another fullerene molecule, for example, any one of the fullerenes represented by Cₙ (n=70, 76, 78, . . . ), the same advantage of increased proton conductivity will be obtained as in the above embodiment based on C₆₀. The proton dissociable group is not limited to —OH group either. If the proton dissociable group is —OH, —OSO₃H, —COOH, —SO₃H or —OPO(OH)₂ instead of —OH, the same advantage of increased proton conductivity will be ensured.

Third Illustrative Embodiment

For a fullerene derivative to function as a good proton conductor, it preferably takes a solid structure where members of the fullerene derivative are densely packed. FIG. 10(a) illustrates a proton conductor comprised of a polymerized endohedral fullerene obtained by polymerizing an inventive endohedral fullerene doped with an atom whose electronegativity is equal to or lower than 1 by using an aromatic group consisting of two benzene rings as a linker. FIG. 10(b) illustrates a proton conductor comprised of a polymerized endohedral fullerene derivative obtained by chemically modifying an inventive endohedral fullerene doped with an atom whose electronegativity is equal to or higher than 3 by means of a proton dissociable group and then polymerizing the resulting endohedral fullerene derivative by using an aromatic group consisting of two benzene rings as a linker.

The manufacture of a polymerized endohedral fullerene consists of reacting at first an endohedral fullerene with a halogen atom to produce an endohedral fullerene halide. For example, an Na doped fullerene is allowed to react with bromine in carbon tetrachloride to produce an endohedral fullerene bromide. The endohedral fullerene bromide is then reacted, in the presence of a Lewis acid-based catalyst, with an aromatic group bearing compound having a general formula:
Ar¹—(CH₂)ₙ—Ar²,
(where n is an integer chosen from 0 to 5, and Ar¹ and Ar² are substituted or unsubstituted aryl groups which may be the same or different from each other), so that the bromide is substituted by the aromatic group. This reaction results in the production of a polymerized endohedral fullerene derivative in which endohedral fullerenes are linked together through the aromatic groups. (Patent Document 2).

FIGS. 10(a) and 10(b) depict a polymerized endohedral fullerene derivative where endohedral fullerene derivatives are linked together to spread on a two-dimensional space. However, a polymerized endohedral fullerene derivative may be used where endohedral fullerene derivatives are linked together to spread in a three-dimensional space. With a proton conductor comprised of a polymerized endohedral fullerene derivative, it is possible to control the location of sites responsible for the transfer of protons and their number more effectively as compared with a proton conductor where members of the endohedral fullerene derivative are physically packed without undergoing any polymerization, and it is also possible to produce a membrane greater in mechanical strength.

(Electric Generation of a Fuel Cell Working on Other Operation Principles)

FIG. 11(a) illustrates the electric generation of a converted methane type solid polymer-based fuel cell. In addition to a fuel cell which utilizes hydrogen as a fuel, there has been known a fuel cell which utilizes a hydrocarbon gas such as methane as a fuel. A methane conversion type fuel cell uses a methane converter and extracts hydrogen from methane utilizing the reaction cited below:
CH₄+2H₂O-->CO₂+4H₂

FIG. 11(b) illustrates the electric generation of a converted methanol type solid polymer-based fuel cell. Methanol is a liquid, and thus has a higher energy density as compared with hydrogen which is a gas, and can be easily stored. According to a known variation of this mode of electric generation, a methanol converter is used for extracting hydrogen from methanol, and hydrogen thus extracted is supplied to a hydrogen direct transfer type fuel cell. The methanol converter extracts hydrogen from methanol utilizing the reaction cited below:
CH₃OH+H₂O-->CO₂+3H₂.

FIG. 11(c) illustrates the electric generation of a direct methanol type solid polymer-based fuel cell. A methanol conversion type fuel cell tends to be large in volume because it must include a methanol converter, and thus its installment is disadvantageous for an automobile or a battery for a mobile phone where the space is limited. To avoid this inconveniency, the development of a direct methanol type fuel cell (DMFC) which directly utilizes methanol as a fuel is currently under way. Reactions observed in the electrodes of a direct methanol type fuel cell are as follows:

On the anode: CH₃OH+H₂O-->CO₂+6H⁺+6e⁻

On the cathode: O₂+4H⁺+4e⁻-->2H₂O

As described above, the fuel cell can utilize a variety of fuels in addition to hydrogen utilized by a hydrogen direct transfer type fuel cell. The electrolyte membrane including an inventive proton conductor may be applied, in addition to the hydrogen direct transfer type fuel cell, with the same advantage to the other fuel cells mentioned above working on different operation modes, because obviously the migration of protons through the electrolyte membrane of those fuel cells occurs in the same manner as in the hydrogen direct transfer type fuel cell.

(Manufacture of Endohedral Fullerene)

First Illustrative Embodiment

Take, as an example, the manufacture of an endohedral fullerene doped with an atom whose electronegativity is equal to or lower than 1, or an endohedral fullerene doped with an alkali metal. One known method consists of applying a jet of metal vapor against a hot plate heated in a vacuum chamber, thereby generating an ionization plasma, and ejecting a jet of fullerene vapor against the metal plasma current so that endohedral fullerenes can deposit on a substrate placed downstream of the plasma current.

As shown in FIG. 12, an exemplary device for manufacturing endohedral fullerenes by utilizing metal plasma comprises a vacuum chamber, means for forming a plasma current comprised of an atom to be doped, means for introducing fullerenes in the plasma, and a deposition substrate 17 placed downstream of the plasma current. The means for forming a plasma current of alkali metal comprises a hot plate 13 and an oven 14 for vaporizing alkali metal. When vaporization oven 14 ejects the vapor of an alkali metal to be doped, plasma is generated as a result of contact ionization. The plasma thus generated is entrapped in a uniform magnetic field (B=2 to 7 kG) formed by means of electromagnetic coils 12 along the longitudinal axis of vacuum chamber 11, and the plasma turns into current flowing from hot plate 13 towards deposition substrate 17. The means for introducing fullerenes into plasma comprises an oven 15 for sublimating fullerenes, and a resublimation cylinder 16. When the vapor of fullerenes consisting of sublimated C₆₀ and others from the fullerene sublimating oven 15 is applied through the resublimation cylinder 16 to the plasma current, C₆₀, because of its high electronegativity, readily seizes an electron in the plasma, thereby turning into a negative ion of C₆₀. If sodium is used as the alkali metal, then following reactions will occur:
Na-->Na⁺+e⁻
C₆₀+e⁻-->C₆₀

Then the plasma current will become a plasma current comprised of positive ions of alkali metal, negative ions of fullerenes, and residual electrons. When the deposition substrate 17 placed downstream of plasma current is given a positive bias voltage, the positive ion of alkali metal having a small mass becomes retarded while the negative ion of fullerenes having a large mass is accelerated, so that interaction between the positive ion of alkali metal and the negative ion of fullerenes is enhanced, thereby promoting the enclosure of metal ion by the fullerenes. The endohedral fullerenes thus produced deposit on the deposition substrate.

Second Illustrative Embodiment

Take, as an example, the manufacture of an endohedral fullerene doped with an atom whose electronegativity is equal to or higher than 3, or an endohedral fullerene doped with fluorine. One known method is an RF induction plasma method which consists of introducing a material gas of CF₄ into a vacuum chamber, and exciting particles constituting the material gas by passing electric current through an RF induction coil 24 wound around the vacuum chamber, thereby generating plasma comprised of ions such as CF₃⁺, F⁻, etc., and electrons.

The plasma thus generated is entrapped in a uniform magnetic field (B=2 to 7 kG) formed by means of electromagnetic coils 22 along the longitudinal axis of vacuum chamber 21, and the plasma turns into current flowing from the site of its generation towards deposition substrate 27. By applying a positive bias voltage to the grid electrode 30 across which the plasma passes, it is possible to selectively pass only negatively charged particles such as electrons and fluorine ions. Electrons, being accelerated by the positive bias voltage applied to the grid electrode, come to have an energy of 10 eV or higher, and collide violently with fullerene molecules ejected by fullerene sublimating oven 25. Then, the electrons expel electrons orbiting round fullerene molecules, thereby producing a positive ion of C₆₀⁺. C₆₀⁺ and F⁻ constituting the plasma react with each other to form fluorine doped fullerenes which deposit on the deposition substrate 27.

(Manufacture of a Proton Conductor (Attachment of Proton Dissociable Group))

A method for attaching a proton dissociable group such as —OH to a fluorine doped fullerene is disclosed, for example, in Non-patent Document 2. The method consists of reacting bromine with fluorine-doped fullerene in carbon tetrachloride, to produce endohedral fullerene bromide. The endohedral fullerene bromide thus produced is allowed to react, in an inert solvent of o-dichlorobenzene to which is added AlCl₃ as a Lewis acid-based catalyst, with a hydroxide (NaOH) at room temperature to produce F@C₆₀(OH)₂.

(Gas Detector)

The applicable field of a proton conductor of the invention is not limited to the fuel cell. Let's take, as an example, a laminated membrane comprising an inventive proton conductor inserted between an anode catalyst and a cathode catalyst. If hydrogen gas is supplied to the anode catalyst, the resistance between an anode in contact with the anode catalyst and a cathode in contact with the cathode catalyst is reduced because then protons are amply supplied to the proton conductor. If a certain voltage is applied between the anode and the cathode, and electric current passing through the laminated membrane is monitored, it is possible to sensitively detect the presence of hydrogen: when the concentration of hydrogen is high, the value of electric current becomes high, while when the concentration of hydrogen is low, the value of electric current becomes low. Thus, if a laminated membrane incorporating an inventive proton conductor is applied to the manufacture of a gas detector, it will be possible to produce a highly sensitive hydrogen sensor having a very simple structure. The gas to be detected is not limited to hydrogen, may include a hydrocarbon gas such as methane, methanol, ethanol, etc. Detection of a hydrocarbon gas will be possible by attaching a converter chosen as appropriate according to a hydrocarbon gas to be detected to a hydrogen gas detector.

FIG. 13(a) is a sectional view of a gas detection unit of a hydrogen gas detector incorporating a proton conductor of the invention. The unit comprises a polymer-based electrolyte membrane 55 functioning as a proton conductor inserted between an anode catalyst 54 and a cathode catalyst 56 which are sandwiched from outside by porous supports 53, 57. The assembly is placed, for example, in a cylindrical tube. The assembly or a laminated membrane is placed with respect to the tube such that anode catalyst 54 and porous support 53 faces a probe opening 51 while cathode catalyst 56 and porous support 57 faces the opposite opening or reference opening 52. Air containing oxygen is introduced from reference opening 52 opposite to porous support 57 into the tube while gas to be detected is introduced from probe opening 51 opposite to porous support 53 into the tube. The tube is not limited to a cylinder in its shape but may take any shape. Anode catalyst 54 and cathode catalyst 56 are connected to respective electrodes so that an anode lead 58 and a cathode lead 59 connected electrically to anode and cathode respectively can be withdrawn from the tube to outside. A voltage is applied between the electrodes via respective leads, and electric current passing through polymer-based electrolyte membrane 55 is monitored.

When hydrogen gas is not supplied via opening 51 to porous support 53, protons supplied to polymer-based electrolyte membrane 55 are sparse. Since then carriers bearing charge are very few, the resistance between the leads 58, 59 is large. On the other hand, when hydrogen gas is supplied to porous support 53, protons supplied to polymer-based electrolyte membrane 55 are plentiful. Since then carriers bearing charge are numerous, the resistance between the leads 58, 59 becomes small. The resistance in question varies depending on the concentration of hydrogen to be tested. Thus, this detector can not only detect the presence of hydrogen but also determine the concentration of hydrogen.

As seen above with respect to fuel cells, the detector can convert, by employing a converter, a hydrocarbon gas into hydrogen for detection, or can directly convert a hydrocarbon gas into proton like DMFC for detection. Accordingly, it is possible by using a detector incorporating a proton conductor of the invention not only to detect a hydrocarbon gas such as methane, methanol, ethanol, etc., as well as hydrogen, but also to determine the concentration of the hydrocarbon gas.

FIG. 13(b) represents a schematic overview of a first embodiment of a gas detector incorporating a proton conductor of the invention. According to this embodiment, Gas detection unit 61 is located at the tip of the cylinder, the value of electric current passing through a laminated membrane acting as a gas sensor, or a signal obtained by processing the value is displayed on a display unit 62. The detector shown in FIG. 13(b) displays the concentration of gas on an analog scale. However, it is also possible by adding an analog/digital converter to convert the analog value into a corresponding digital value to be displayed.

FIG. 13(c) represents a schematic overview of a second embodiment of a gas detector incorporating a proton conductor of the invention. The detector has a flange 65 attached to the distal end of a gas detection unit 66. The detector can determine the concentration of test gas under the combinational use of vacuum equipment.

(Leak Detector)

A hydrogen sensor incorporating a proton conductor of the invention can detect leak of vacuum equipment by using hydrogen as a probe gas. A gas detector incorporating a proton conductor of the invention can detect not only hydrogen but also a hydrocarbon gas such as methane, methanol, ethanol, etc. Accordingly a gas detector of the invention can be utilized not only for checking a gas range and gas distribution system for leak of coal gas or propane gas, but also for detecting alcohol in the breath from a driver suspected of alcohol drinking. In all these applications, the gas detector of the invention is advantageous in many respects, for example, in low production cost, compactness, small weight, and detection sensitivity.

(Conventional Leak Detector)

FIG. 16 is a block diagram for illustrating the organization of a conventional helium leak detector. A vacuum unit 105 to be tested is connected via a distributor pipe 102 and valve 103 to a rotary pump 106. A leak detector 101 is fixed via flange to a pipe extending between vacuum unit 105 and rotary pump 106. The leak detector 101 comprises a diffusion pump 108, a vacuum meter 107, and a mass spectrometer. The mass spectrometer comprises an ion source 109, analysis tube 110, ion collector 111, amplifier 112 and power supply 113. The mass spectrometer is a detector whose sensitivity is selectively tuned to helium. When helium gas is blown from outside against the vacuum unit 105, and if there is a leak spot on the vacuum unit, helium will enter through the spot into the vacuum unit and flow through distributor pipe into the mass spectrometer. Thus, with this leak detector, it is possible not only to detect leak of the vacuum unit, but also to locate the leak site. However, helium is expensive and the mass spectrometer is very complicated. Thus, it has been demanded to develop a novel leak detector where a more inexpensive probe gas is used instead of helium, and a smaller, lighter analyzer is employed instead of a mass spectrometer.

(Inventive Leak Detector)

FIG. 14(a) illustrates an embodiment where a gas detector of the invention is used for checking a vacuum unit for leak. Diluted hydrogen gas is introduced into the interior of the vacuum unit, and the gas detector 72 is moved around the external surfaces of the vacuum unit to see whether any leak site is present.

FIG. 14(b) illustrates an embodiment where a gas detector of the invention is used for checking a gas range for leak. By moving the gas detector 74 around the gas range 73, one can check the gas range for leak by detecting methane of the gas.

Leak checking based on hydrogen gas is more advantageous in following points than conventional leak checking based on helium gas.

(1) It is possible to use hydrogen which is cheaper than helium. Like helium, the content of hydrogen in air is so small that its background noise during measurement is trivial. Like helium, hydrogen molecules have such a small diameter that they can easily invade through a leak spot, even if the spot is tiny. Thus, a detector using hydrogen will allow the highly precise detection of leak.

(2) The need for a sophisticated, expensive mass spectrometer can be obviated. It becomes possible to produce a leak detector which is compact, light, portable, and cheap. Since the leak detector is light in weight, it is possible to readily detect the leak of a vacuum unit by internal pressurization as well as by probe gas flushing: internal pressurization consists of introducing probe gas in the interior of a vacuum unit and moving the detector around the unit to check for leak, while probe gas flushing which is commonly adopted consists of applying probe gas onto a vacuum unit from outside to check for leak.

FIGS. 15(a) and 15(b) illustrate the principle underlying the test for checking a vacuum unit for leak. FIG. 15(a) illustrates how leak can be checked by probe gas flushing. FIG. 15(b) illustrates how leak can be checked by internal pressurization.

To check for leak by probe gas flushing, a subject unit 81 to be tested represented, for example, by a vacuum unit or ultra-vacuum unit is evacuated with a rotary pump 83 as shown in FIG. 15(a). To a connector pipe 82 connecting rotary pump 83 to subject unit 81, a gas detector 85 is fixed through a flange 84. A probe gas flushing unit 86 is activated to flush probe gas represented by hydrogen onto the surface of subject unit 81. The content of hydrogen in the probe gas should be adjusted to be equal to or less than 7%, for fear that explosion might occur. If there is any leak in the subject unit 81, a gas detector 85 can detect hydrogen crept in the subject unit 81, thereby confirming the presence of leak. At the same time, the detector can locate the site of leak.

To check for leak by internal pressurization, probe gas represented by hydrogen is supplied through a distributor pipe 87 to a subject unit 88 as shown in FIG. 15(b). If there is any leak in the subject unit 88, hydrogen will escape from the subject unit 88 to outside. Then, a gas detector 89 can confirms the presence of leak, and locate the site of leak at the same time.

### EXAMPLES

The present invention will be detailed below with reference to examples, but it should be understood that the present invention is not limited in any way to those examples.

### Manufacture Example 1

(Production of Li Doped Fullerenes)

To manufacture Li doped fullerenes, used is a device having a structure consisting of a cylindrical vessel made of stainless steel with electromagnetic coils arranged thereon as shown in FIG. 12(a). Li to serve as a starting material was obtained from Aldrich, and was not purified in terms of the content of isotopes. C₆₀ to serve as another starting material was obtained from Frontier Carbon. The vacuum vessel was evacuated to achieve a vacuum of 4.2×10⁻⁵ Pa. The electromagnetic coils were activated to generate a magnetic field of 0.2 T. Solid Li was transferred to a dopant atom sublimating oven which was then heated to 480° C. to sublimate Li into gas. The Li gas thus produced was guided through gas conducting tube heated at 500° C. into the vessel. The gas was applied as a jet stream onto a thermal ionization plate heated at 2500° C. The Li vapor undergoes inonization on the surface of the thermal ionization plate to produce plasma current comprising positive ions of Li and electrons. Into the plasma current, was introduced C₆₀ vapor which was obtained by sublimating C₆₀ in a fullerene oven heated at 610° C. A bias voltage of +10V was applied to a deposition substrate placed to come into contact with plasma current so that a film including endohedral fullerenes can be formed on the surface of deposition substrate. Fullerenes were allowed to deposit for about 1 hour. Then, a film having a thickness of 0.9 μm was formed.

### Manufacture Example 2

(Isolation and Purification of Li Doped Fullerenes)

The film thus formed was separated from the deposition plate, and ground into a powder which was then dissolved to a solvent consisting of carbon disulfide. Then, Li doped fullerenes were separated by HPLC from fullerenes not doped with Li.

### Manufacture Example 3

(Production of Proton Conductor Comprised of Li Doped) Fullerenes

A 110 mg of a powder of Li doped fullerenes having a purity of about 90% was transferred to a pressing machine so that the powder was compressed under a unilaterally applied pressure of 6 t/cm² to give a round disc having a diameter of 20 mm. The powder of Li doped fullerenes could be readily reduced to a disc shape without requiring the addition of a binder resin. The thickness of the disc was about 400 μm.

### Manufacture Example 4

(Production of Polymerized Li Doped Fullerenes)

To 60 ml of ortho-dichlorobenzene (ODCB) to which 50 mg of Li doped fullerenes obtained in manufacture example 5 had been dissolved, 5 ml of ODCB solution containing 150 mg of iodine monobromide was added. This mixture solution was stirred, and left at room temperature for 3 days. Then, the mixture solution was removed of solvent and iodine under a reduced pressure so that 60 mg of a residue could be obtained. The yield was washed with pentane, and heated to 60° C. after washing. The pressure was reduced to 0.1 mmHg, and the yield was maintained at 60° C. for 5 hours to give Li@C₆₀Br₆.

Next, Li doped fullerene bromide thus produced was allowed to react with phenol (C₆H₄OH) and biphenyl in ODCB in the presence of a Lewis acid-based catalyst (AlCl₃) to give a polymer in which Li doped fullerenes or Li@C₆₀ molecules serving as a monomer unit are linked together through a biphenyl group. Polymerized Li doped fullerenes thus produced was processed to be disc-shaped. Thus, a disc having a thickness of about 400 μm was obtained.

Manufacture of Fuel Cell

To serve as a reference, fuming sulfuric acid was added to fullerenes C₆₀, which were then hydrolyzed to give fullerenol C₆₀(OH)ₙ. C₆₀(OH)ₙ corresponds to a conventional proton conductor obtained by adding a proton dissociable group OH to hollow fullerene. A 110 mg of a powder of C₆₀(OH)ₙ was transferred to a pressing machine so that the powder was compressed under a unilaterally applied pressure of 6 t/cm² to give a round disc having a diameter of 20 mm.

Each of the three kinds of discs consisting of Li doped fullerenes, polymerized Li doped fullerenes, or fullerenol was inserted between an anode and a cathode to produce a disc-shaped fuel cell having a diameter of about 20 mm. They were named cell 1, cell 2 and cell 3. Each of the anode and cathode was obtained by coating a carbon carrier in which platinum catalyst was dispersed highly densely on the surface of a porous support.

Measurement of the Dependency of Electromotive Force on Output Current

From each of cells 1, 2 and 3, five fuel batteries were prepared each consisting of 16 cells arranged in parallel. For each fuel battery, hydrogen is supplied at a certain rate to the fuel electrode, while the air electrode is allowed to come in contact with air, to allow electricity to be generated. The output current conducted from the battery was varied from 0 to 10A, and voltage varyed in association was monitored. The measurement was performed on five fuel batteries prepared from each kind of fuel cell. The measurement data listed below represent the averaged electromotive forces obtained from each type of fuel battery expressed in relative terms with respect to cell 1-based fuel batteries whose electromotive force was taken as 1 (unit) when the output current was 0. The voltage drop observed when the output current is increased is less conspicuous for the fuel battery incorporating Li doped fullerene or polymerized Li doped fullerene than for the fuel battery based on hollow fullerene.

Average Electromotive Force

| | Material of | | | |
|---|---|---|---|---|
| Name of | electrolyte | Output current (A) | | |
| cell | membrane. | 0 | 5 | 10 |
| Cell 1 | Li doped fullerene | 1 | 0.79 | 0.63 |
| Cell 2 | Li doped fullerene | 1.02 | 0.81 | 0.62 |
| | polymer | | | |
| Cell 3 | Fullerenol | 0.99 | 0.65 | 0.49 |

### INDUSTRIAL APPLICABILITY

(1) With regard to a proton conductor comprised of an endohedral fullerene derivative obtained by chemically modifying an endohedral fullerene doped with an atom whose electric negativity is equal to or higher than 3, by means of a proton dissociable group such as —OH, —OSO₃H, —COOH, —SO₃H, or —OPO(OH)₂, when such a proton conductor is used as an electrolyte membrane of a fuel cell, dissociation of proton from a proton dissociable group occurs easily because proton is attracted by the dopant atom, but the attractive force exerted by the dopant atom is small because of the negatively charged dopant atom being enclosed in the cage of fullerene. Because of these properties, protons can freely move in the electrolyte membrane which results in the increased proton conductivity of the electrolyte membrane.

(2) With regard to a proton conductor comprised of an endohedral fullerene doped with an atom whose electronegativity is equal to or lower than 1, the dopant atom will become a positive ion by giving off an electron to the fullerene cage. The fullerene cage becomes negatively charged because of receiving an electron. However, the electron is not localized upon a specific carbon, and thus the attractive force exerted by the fullerene cage towards proton is comparatively weak. Therefore, protons can move freely being driven by a comparatively low thermal energy through the electrolyte membrane where proton conducting elements are densely packed, which results in the increased proton conductivity of the electrolyte membrane.

(3) With regard to a proton conductor comprised of a polymerized endohedral fullerene or a polymerized endohedral fullerene derivative obtained by polymerizing an endohedral fullerene or an endohedral fullerene derivative, it is excellent in its mechanical strength.

(4) A fuel battery incorporating an electrolyte membrane based on a proton conductor of the invention is more advantageous than a conventional battery incorporating a fluorine resin-based electrolyte membrane, because the inventive membrane obviates the need for moisturization, and enables the production of a thinner membrane capable of working over a wider temperature range, and further reduces the internal resistance sufficiently low as to inhibit the dropping of voltage even if big current is extracted.

(5) According to a gas detector incorporating an electrolyte membrane based on a proton conductor of the invention, it is possible to determine the concentration of hydrogen or hydrocarbon at high sensitivity.

(6) According to a leak detector incorporating an electrolyte membrane based on a proton conductor of the invention, it is possible to check a vacuum unit or gas range for leak at high sensitivity by using, for example, hydrogen as probe gas.

1. An endohedral fullerene derivative obtained by chemically modifying an endohedral fullerene doped with an atom whose electronegativity is 3 or higher, by means of a proton dissociable group.

2. The endohedral fullerene derivative as described in claim 1 wherein the proton dissociable group is any one selected from the group comprising —OH, —OSO₃H, —COOH, —SO₃H, and —OPO(OH)₂.

3. A proton conductor comprised of an endohedral fullerene derivative as described in claim 1.

4. A proton conductor comprised of an endohedral fullerene doped with an atom whose electronegativity is equal to or less than 1.

5. A proton conductor comprised of a polymerized endohedral fullerene derivative obtained by polymerizing an endohedral fullerene derivative as described in claim 3.

6. A fuel battery comprising a stack of cells each comprising a fuel electrode, an electrolyte membrane including a proton conductor as described in claim 3 and an air electrode.

7. A gas detector having a gas detection unit comprising a stack of cells each comprising an anode, an electrolyte membrane including a proton conductor as described in claim 3, and a cathode.

8. A method for determining the concentration of gas such as hydrogen or hydrocarbon gas using a gas detector as described in claim 7.

9. A leak detector having a gas detection unit comprising a stack of cells each comprising an anode, an electrolyte membrane including a proton conductor as described in claim 3, and a cathode.

10. Leak detection method for checking whether any leak occurs in a device to be tested and for identifying the site of leak if any leak is detected, the method comprising employing hydrogen as a probe gas, and using a leak detector as described in claim 9.

11. A proton conductor comprised of an endohedral fullerene derivative as described in claim 2.

12. A proton conductor comprised of a polymerized endohedral fullerene derivative obtained by polymerizing an endohedral fullerene derivative as described in claim 4.

13. A fuel battery comprising a stack of cells each comprising a fuel electrode, an electrolyte membrane including a proton conductor as described in claim 4, and an air electrode.

14. A fuel battery comprising a stack of cells each comprising a fuel electrode, an electrolyte membrane including a proton conductor as described in claim 5, and an air electrode.

15. A gas detector having a gas detection unit comprising a stack of cells each comprising an anode, an electrolyte membrane including a proton conductor as described in claim 4, and a cathode.

16. A gas detector having a gas detection unit comprising a stack of cells each comprising an anode, an electrolyte membrane including a proton conductor as described in claim 5, and a cathode.

17. A leak detector having a gas detection unit comprising a stack of cells each comprising an anode, an electrolyte membrane including a proton conductor as described in claim 4, and a cathode.

18. A leak detector having a gas detection unit comprising a stack of cells each comprising an anode, an electrolyte membrane including a proton conductor as described in claim 5, and a cathode.
